(19) United States
(12) Patent Application Publication
(10) Pub. No.: US 2010/0124539 A1
(43) Pub. Date: May 20, 2010

(54) COSMETIC PREPARATIONS

(75) Inventor: Gerald L. Hanson, Kingston, WA (US)

Correspondence Address: BARNES & THORNBURG LLP, 11 SOUTH MERIDIAN, INDIANAPOLIS, IN, 46204, US

(73) Assignee: HANSON MEDICAL, INC., Kingston, WA (US)

(21) Appl. No.: 12/691,010

(22) Filed: Jan. 21, 2010

Related U.S. Application Data
(63) Continuation-in-part of application No. 11/868,773, filed on Oct. 8, 2007.
(60) Provisional application No. 61/265,382, filed on Dec. 1, 2009.
(60) Provisional application No. 60/852,073, filed on Oct. 16, 2006.

(51) Int. Cl. A61K 8/58; A61K 8/81; A61K 8/49; A61Q 19/02; A61Q 19/00

(52) U.S. Cl. 424/62; 424/78.03; 514/474

(57) ABSTRACT

According to an aspect of the invention, a cosmetic preparation comprises about 1% to about 80% by weight of the finished preparation a first silicone fluid having a kinematic viscosity in the range of about 10,000 centistokes (cs) to about 15,000 cs, about 1% to about 80% by weight of the finished preparation a second silicone fluid having a kinematic viscosity in the range of about 25 cs to about 75 cs, about 1% to about 80% by weight of the finished preparation a third silicone fluid having a kinematic viscosity in the range of about 1 cs to about 10 cs, about 0.25% to about 80% by weight of the finished preparation an ascorbic acid ester, about 0.1% to about 1.0% by weight of the finished preparation hexylresorcinol, optionally about 1% to about 80% by weight of the finished preparation vinyl silicone crosspolymer, optionally about 0.25% to about 10% by weight of the finished preparation a fragrance-providing composition, and optionally about 0.1% to about 1.0% by weight of the finished preparation other skin lightening ingredients. Also provided are uses of the preparations.

### CROSS-REFERENCE TO RELATED APPLICATIONS

This application is a continuation-in-part of U.S. Ser. No. 11/868,773, filed Oct. 8, 2007. U.S. Ser. No. 11/868,773 claims the benefit of the Oct. 16, 2006 filing date of U.S. Ser. No. 60/852,073. The disclosures of both U.S. Ser. No. 60/852,073 and U.S. Ser. No. 11/868,773 are hereby incorporated herein by reference.

### FIELD OF THE INVENTION

This invention relates generally to cosmetic preparations. It is disclosed in the context of a cosmetic preparation for application to, for example, hypertrophic scars, keloid scars and erythema. However, it is believed to be useful in other applications as well.

### BACKGROUND OF THE INVENTION

Cosmetic preparations are known. There are, for example, the cosmetic preparations described in U.S. Pat. No. 5,741,509 and the references cited therein. This listing is not intended to be a representation that a complete search of all relevant art has been made, or that no more pertinent art than that listed exists, or that the listed art is material to patentability. Nor should any such representation be inferred.

### DISCLOSURE OF THE INVENTION

According to an aspect of the invention, a cosmetic preparation comprises about 1% to about 80% by weight of the finished preparation a first silicone fluid having a kinematic viscosity in the range of about 10,000 centistokes (cs) to about 15,000 cs, about 1% to about 80% by weight of the finished preparation a second silicone fluid having a kinematic viscosity in the range of about 25 cs to about 75 cs, about 1% to about 80% by weight of the finished preparation a third silicone fluid having a kinematic viscosity in the range of about 1 cs to about 10 cs, about 0.25% to about 80% by weight of the finished preparation an ascorbic acid ester, about 0.1% to about 1.0% by weight of the finished preparation hexylresorcinol, optionally about 1% to about 80% by weight of the finished preparation vinyl silicone crosspolymer, optionally about 0.25% to about 10% by weight of the finished preparation a fragrance-providing composition, and optionally about 0.1% to about 1.0% by weight of the finished preparation other skin lightening ingredients.

Illustratively according to this aspect of the invention, the cosmetic preparation comprises about 1% to about 80% by weight of the finished preparation tetrahexyldecyl ascorbate.

Further illustratively according to this aspect of the invention, the cosmetic preparation comprises about 0.5% to about 10% by weight of the finished preparation tetrahexyldecyl ascorbate.

Illustratively according to this aspect of the invention, the cosmetic preparation comprises about 1% to about 80% by weight of the finished preparation a first silicone fluid having a kinematic viscosity about 12,500 cs.

Further illustratively according to this aspect of the invention, the cosmetic preparation comprises about 50% to about 80% by weight of the finished preparation a first silicone fluid having a kinematic viscosity about 12,500 cs.

Illustratively according to this aspect of the invention, the cosmetic preparation comprises about 1% to about 80% by weight of the finished preparation a second silicone fluid having a kinematic viscosity about 50 cs.

Further illustratively according to this aspect of the invention, the cosmetic preparation comprises about 20% to about 40% by weight of the finished preparation a second silicone fluid having a kinematic viscosity about 50 cs.

Illustratively according to this aspect of the invention, the cosmetic preparation comprises about 1% to about 80% by weight of the finished preparation a third silicone fluid having a kinematic viscosity about 5 cs.

Further illustratively according to this aspect of the invention, the cosmetic preparation comprises about 20% to about 40% by weight of the finished preparation a third silicone fluid having a kinematic viscosity about 5 cs.

### DETAILED DESCRIPTIONS OF ILLUSTRATIVE EMBODIMENTS

The invention may best be understood by referring to the following detailed description and examples which illustrate the invention. A cosmetic preparation is prepared by combining about 1% to about 80% by weight of the finished preparation a first silicone fluid having a kinematic viscosity in the range of about 10,000 cs to about 15,000 cs, about 1% to about 80% by weight of the finished preparation a second silicone fluid having a kinematic viscosity in the range of about 25 cs to about 75 cs, about 1% to about 80% by weight of the finished preparation a third silicone fluid having a kinematic viscosity in the range of about 1 cs to about 10 cs, about 0.1% to about 1.0% by weight of the finished preparation hexylresorcinol, optionally about 1% to about 80% by weight of the finished preparation vinyl silicone crosspolymer, optionally about 0.25% to about 10% by weight of the finished preparation a fragrance-providing composition, and optionally about 0.1% to about 1.0% by weight of the finished preparation other skin lightening ingredients.

Illustratively, the silicones are in the form of dimethicones, that is, silicone oils comprising dimethylsiloxane polymers, usually incorporated into petrolatum bases or other nongreasy preparations. Such silicones are generally used for the protection of normal skin against, for example, industrial, skin irritants. Illustratively, the vinyl silicone crosspolymer is in the form of vinyl dimethicone crosspolymer.

Illustratively, the about 1% to about 80% by weight of the finished preparation of a first silicone fluid having a kinematic viscosity in the range of about 10,000 cs to about 15,000 cs comprises silicone fluid having a kinematic viscosity of about 12,500 cs. Illustratively, the about 1% to about 80% by weight of the finished preparation of a first silicone fluid having a kinematic viscosity in the range of about 10,000 cs to about 15,000 cs comprises Dow Corning® 200® fluid, kinematic viscosity about 12,500 cs, available from Dow Corning Corporation, Midland, Michigan.

Illustratively, the about 1% to about 80% by weight of the finished preparation of a second silicone fluid having a kinematic viscosity in the range of about 25 cs to about 75 cs comprises silicone fluid having a kinematic viscosity of about 50 cs. Illustratively, the about 1% to about 80% by weight of the finished preparation of a second silicone fluid having a kinematic viscosity in the range of about 25 cs to about 75 cs comprises Dow Corning® 200® fluid, kinematic viscosity about 50 cs, available from Dow Corning Corporation, Midland, Mich.

Illustratively, the about 1% to about 80% by weight of the finished preparation a third silicone fluid having a kinematic viscosity in the range of about 1 cs to about 10 cs comprises silicone fluid having a kinematic viscosity of about 5 cs. Illustratively, the about 1% to about 80% by weight of the finished preparation of a third silicone fluid having a kinematic viscosity in the range of about 1 cs to about 10 cs comprises Dow Corning® 200® fluid, kinematic viscosity about 5 cs, available from Dow Corning Corporation, Midland, Mich.

Illustratively, the about 1% to about 80% by weight of the finished preparation vinyl silicone crosspolymer comprises about 1% to about 80% by weight of the finished preparation Dow Corning® 2502® fluid, available from Dow Corning Corporation, Midland, Mich.

Illustratively, the about 1% to about 80% by weight of the finished preparation ascorbate ester comprises about 1% to about 80% by weight of the finished preparation tetrahexyldecyl ascorbate.

Illustratively, the cosmetic preparation may contain about 0.25% to about 10%, or about 0.25% to about 5%, by weight of the finished preparation a fragrance-providing composition. An example of a fragrance-providing composition is orange oil. An illustrative orange oil is Bell Flavors & Fragrances 10-fold natural orange oil.

Hexylresorcinol is present in the compositions in the amount of between about 0.1% and 1.0% by weight of the finished preparation. Hexylresorcinol is a skin lightening agent, and helps to prevent the darkening of scars, for example, by inhibiting tyronase and peroxidase H₂O₂. Particularly in those of Asian, Mediterranean, Latin, or Indian descent, the pigmentation of scar tissue often becomes darker than normal tissue. Hexylresorcinol is sold under the name Synovea® HR by Sytheon Ltd. of New Jersey. Additional skin lightening ingredients may be included, and examples of such are hydroquinone, kojic acid, arbutin, and vitamin C derivatives such as ascorbyl glucoside, magnesium ascorbyl phosphate, and sodium ascorbyl phosphate. The additional skin lightening ingredients are present in the preparations in the amount of between 0.1 about 0.1% and 1.0% by weight of the finished preparation.

### EXAMPLE 1

In a specific, but only illustrative, example, about 69% by weight of the finished preparation about 12,500 cs silicone fluid, about 14% by weight of the finished preparation about 50 cs silicone fluid, about 14% by weight of the finished preparation about 5 cs silicone fluid, about 1% by weight of the finished preparation vinyl silicone crosspolymer, about 1% by weight of the finished preparation tetrahexyldecyl ascorbate, and about 1.0% by weight of the finished preparation hexylresorcinol are blended to create a cosmetic preparation for application to, and for use in treatment of, for example, hypertrophic scars, keloid scars and erythema.

### EXAMPLE 2

In another example, about 67.5% by weight of the finished preparation about 12,500 cs silicone fluid, about 15% by weight of the finished preparation about 50 cs silicone fluid, about 14.5% by weight of the finished preparation about 5 cs silicone fluid, about 1% by weight of the finished preparation silicone/vinyl silicone crosspolymer, about 0.5% by weight of the finished preparation tetrahexyldecyl ascorbate, about 1.0% by weight of the finished preparation hexylresorcinol, and about 0.5% by weight of the finished preparation orange oil are blended to create a cosmetic preparation for application to, and for use in treatment of, for example, hypertrophic scars, keloid scars and erythema. An illustrative orange oil is Bell Flavors & Fragrances 10-fold natural orange oil.

What is claimed is:

1. A cosmetic preparation comprising about 1% to about 80% by weight of the finished preparation a first silicone fluid having a kinematic viscosity in the range of about 10,000 cs to about 15,000 cs, about 1% to about 80% by weight of the finished preparation a second silicone fluid having a kinematic viscosity in the range of about 25 cs to about 75 cs, about 1% to about 80% by weight of the finished preparation a third silicone fluid having a kinematic viscosity in the range of about 1 cs to about 10 cs, about 0.25% to about 80% by weight of the finished preparation an ascorbic acid ester, about 0.1% to about 1.0% by weight of the finished preparation hexylresorcinol, optionally about 1% to about 80% by weight of the finished preparation vinyl silicone crosspolymer, optionally about 0.25% to about 10% by weight of the finished preparation a fragrance-providing composition, and optionally about 0.1% to about 1.0% by weight of the finished preparation other skin lightening ingredients.

2. The cosmetic preparation of claim 1 wherein said ascorbic acid ester comprises tetrahexyldecyl ascorbate.

3. The cosmetic preparation of claim 2 wherein said first silicone fluid has a kinematic viscosity of about 12,500 cs.

4. The cosmetic preparation of claim 2 wherein said second silicone fluid has a kinematic viscosity of about 50 cs.

5. The cosmetic preparation of claim 2 wherein said third silicone has a kinematic viscosity of about 5 cs.

6. The cosmetic preparation of claim 2 wherein said ascorbic acid ester comprises about 0.25% to about 10% by weight of the finished preparation tetrahexyldecyl ascorbate.

7. The cosmetic preparation of claim 6 wherein said first silicone fluid has a kinematic viscosity of about 12,500 cs.

8. The cosmetic preparation of claim 6 wherein said second silicone fluid has a kinematic viscosity of about 50 cs.

9. The cosmetic preparation of claim 6 wherein said third silicone fluid has a kinematic viscosity of about 5 cs.

10. The cosmetic preparation of claim 1 comprising about 0.25% to about 10% by weight of the finished preparation an ascorbic acid ester.

11. The cosmetic preparation of claim 10 wherein said first silicone fluid has a kinematic viscosity of about 12,500 cs.

12. The cosmetic preparation of claim 10 wherein said second silicone fluid has a kinematic viscosity of about 50 cs.

13. The cosmetic preparation of claim 10 wherein said third silicone fluid has a kinematic viscosity of about 5 cs.

14. The cosmetic preparation of claim 1 wherein said first silicone fluid has a kinematic viscosity of about 12,500 cs.

15. The cosmetic preparation of claim 1 wherein said second silicone fluid has a kinematic viscosity of about 50 cs.

16. The cosmetic preparation of claim 1 wherein said third silicone fluid has a kinematic viscosity of about 5 cs.

17. The cosmetic preparation of claim 1 further comprising about 0.25% to about 80% by weight of the finished preparation vinyl silicone crosspolymer.

18. The cosmetic preparation of claim 17 wherein said first silicone fluid has a kinematic viscosity of about 12,500 cs.

19. The cosmetic preparation of claim 17 wherein said second silicone fluid has a kinematic viscosity of about 50 cs.

20. The cosmetic preparation of claim 17 wherein said third silicone fluid has a kinematic viscosity of about 5 cs.

21. The cosmetic preparation of claim 17 comprising about 0.25% to about 10% by weight of the finished preparation an ascorbic acid ester.

22. The cosmetic preparation of claim 17 comprising about 0.25% to about 10% by weight of the finished preparation a fragrance-providing composition.

23. The cosmetic preparation of claim 22 comprising about 0.25% to about 5% by weight of the finished preparation orange oil.

24. A cosmetic preparation comprising about 50% to about 80% by weight of the finished preparation a first silicone fluid having a kinematic viscosity in the range of about 10,000 cs to about 15,000 cs, about 20% to about 40% by weight of the finished preparation a second silicone fluid having a kinematic viscosity in the range of about 25 cs to about 75 cs, about 20% to about 40% by weight of the finished preparation a third silicone fluid having a kinematic viscosity in the range of about 1 cs to about 10 cs, about 0.25% to about 10% by weight of the finished preparation vinyl silicone crosspolymer, about 0.25% to about 10% by weight of the finished preparation an ascorbic acid ester, about 0.1% to about 1.0% by weight of the finished preparation hexylresorcinol, optionally about 0.25% to about 10% by weight of the finished preparation a fragrance-providing composition, and optionally about 0.1% to about 1.0% by weight of the finished preparation other skin lightening ingredients.

25. The cosmetic preparation of claim 24 comprising about 0.25% to about 5% by weight of the finished preparation orange oil.

26. A method of treating a hypertrophic scar, a keloid scar, or erythema comprising applying a preparation of claim 1 to said scar or erythema.

27. A method of treating a hypertrophic scar, a keloid scar, or erythema comprising applying a preparation of claim 24 to said scar or erythema.
